(19) United States
(12) Patent Application Publication
(10) Pub. No.: US 2014/0358242 A1
(43) Pub. Date: Dec. 4, 2014

(54) IMPLANT AUGMENT

(71) Applicant: Smith & Nephew, Inc., Memphis, TN (US)

(72) Inventor: Angela Black Mines, Mason, TN (US)

(21) Appl. No.: 14/364,700

(22) PCT Filed: Dec. 13, 2012
(86) PCT No.: PCT/US2012/069573

§ 371 Date: Jun. 12, 2014

Related U.S. Application Data
(60) Provisional application No. 61/570,352, filed on Dec. 14, 2011.

(51) Int. Cl. A61F 2/38

(52) U.S. Cl. CPC A61F 2/38; A61F 2/389; A61F 2/3859
USPC 623/20.32; 623/20.35; 623/20.14

(57) ABSTRACT

An implant augment (12) includes an augment portion (24) and a fixation portion (28) attached to the augment portion (24). The augment portion (24) has a first surface (40) for contacting a cut surface of an articular end of a bone and a second surface (42) for contacting an implant. The fixation portion (28) extends from the augment portion (24) to contact a cortical surface of a shaft of the bone.

### CROSS-REFERENCE TO RELATED APPLICATIONS

This application claims priority to and the full benefit of U.S. Provisional Application Ser. No. 61/570,352, filed Dec. 14, 2011 and entitled “IMPLANT AUGMENT,” the entire contents of which are incorporated herein by reference.

### TECHNICAL FIELD

This document relates to implant augments.

### BACKGROUND

Knee revision surgery, which is also known as revision total knee arthroplasty, is a procedure in which the surgeon removes a previously implanted artificial knee joint, or prosthesis, and replaces it with a new prosthesis. The need for knee revision surgery may be indicated due to loosening of the original prosthesis.

After removal of the old femoral and tibial components of the knee prosthesis, damaged bone is typically scraped off and the bone is reshaped. Augments may be used with the new components of the knee prosthesis to adjust for shortening of the bones due to the additional bone removal.

### SUMMARY

According to one aspect, an implant augment includes an augment portion and a fixation portion attached to the augment portion. The augment portion has a first surface for contacting a cut surface of an articular end of a bone and a second surface for contacting an implant. The fixation portion extends from the augment portion to contact a cortical surface of a shaft of the bone.

Implementations of this aspect may include one or more of the following features.

For example, the fixation portion of the implant defines one or more through holes for receiving fasteners. The through holes are locking holes or non-locking holes. The fixation portion is patient matched to an anatomical shape of a patient's bone. The fixation portion includes a porous in-growth surface. The implant is a femoral augment or a tibial augment. The augment portion defines a through hole. The augment portion is formed from a biocompatible material. The fixation portion includes a surface that enables cement adherence between the fixation portion and the cortical surface of the bone.

According to another aspect, a surgical method includes positioning an augment portion of an implant augment on a cut surface of an articular end of a bone, and fixing a fixation portion of the implant augment to a cortical surface of a shaft of the bone.

Implementations of this aspect may include one or more of the following features.

For example, fixing the fixation portion of the implant augment includes inserting one or more fasteners through one or more openings defined in the fixation portion into the bone. The method includes detaching the fixation portion from the augment portion following installation of the implant augment. The implant augment is a femoral augment. The implant augment is a tibial augment.

The details of one or more implementations are set forth in the accompanying drawings and the description below. Other features, aspects, and advantages will become apparent from the description, the drawings, and the claims.

### DESCRIPTION OF DRAWINGS

FIG. 1 is a perspective view of a knee implant with femoral and tibial augments.

FIG. 2 is a perspective view of a femoral component of the knee implant with a femoral augment.

FIG. 3 is a perspective view of a tibial component of the knee implant with a tibial augment.

FIG. 4 is another perspective view of the femoral component with the femoral augment.

FIGS. 5A-5C are perspective views of the femoral augment.

FIG. 6 is a top view of a base portion of the femoral augment.

FIG. 7 is another perspective view of the tibial component with the tibial augment.

FIG. 8 is a perspective view of the tibial augment

FIG. 9 is a perspective view of a femoral component of the knee implant with another implementation of the femoral augment.

FIG. 10 is a perspective view of another implementation of a femoral augment.

FIG. 11 is a perspective view of a tibial component of the knee implant with another implementation of the tibial augment.

FIG. 12 is a perspective view of another implementation of a tibial augment.

FIG. 13 is a perspective view of a polyaxial opening.

FIGS. 14A-14C are perspectives views of alternative implementations of a femoral augment.

### DETAILED DESCRIPTION

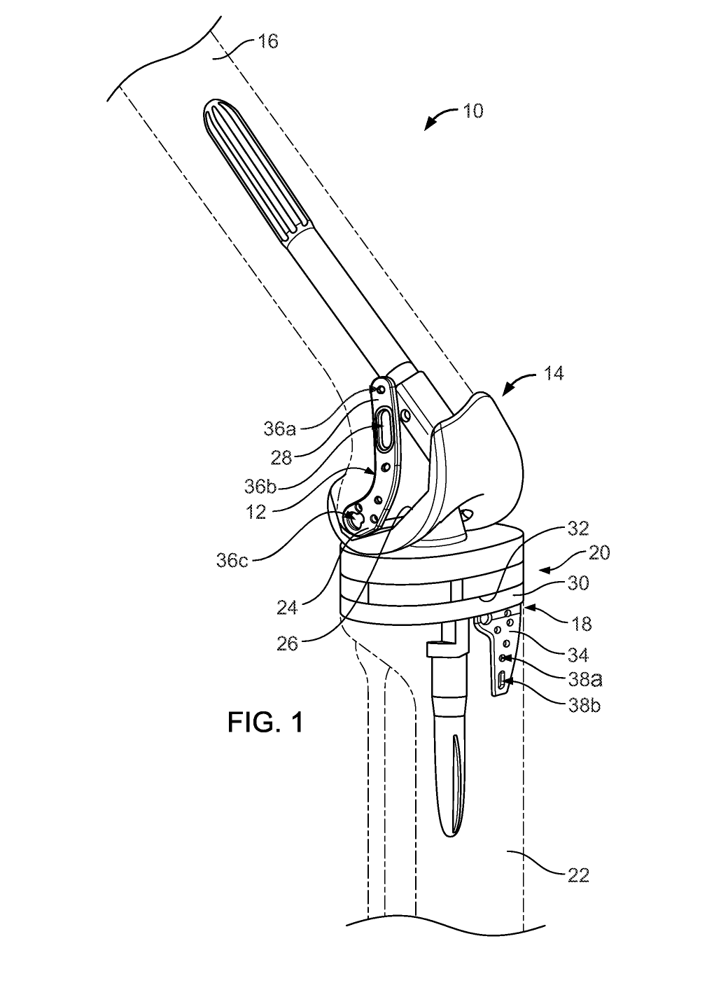

Referring to FIG. 1, during a total knee replacement procedure, particularly a revision procedure, to properly position a knee implant 10 the surgeon may need to include a femoral augment 12 between a femoral component 14 of the knee implant 10 and a femur 16 and/or a tibial augment 18 between a tibial component 20 of the knee implant 10 and a tibia 22. The femoral augment 12 includes an augment portion in the form of a base 24, which is positioned against an inner surface 26 of the femoral component 14, and a fixation portion 28 extending proximally from the base 24. The tibial augment 18 includes an augment portion in the form of a base 30, which is positioned against a face 32 of the tibial component 20, and a fixation portion 34 extending distally from the base 30.

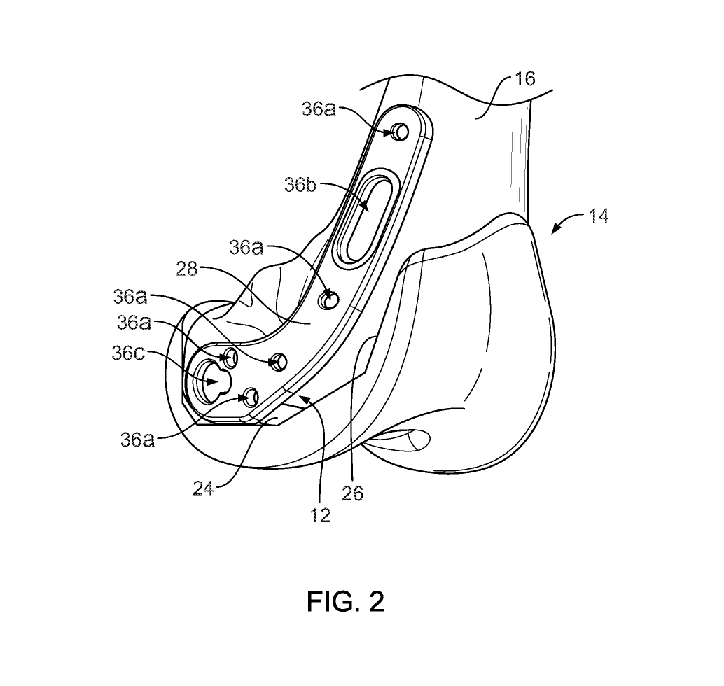

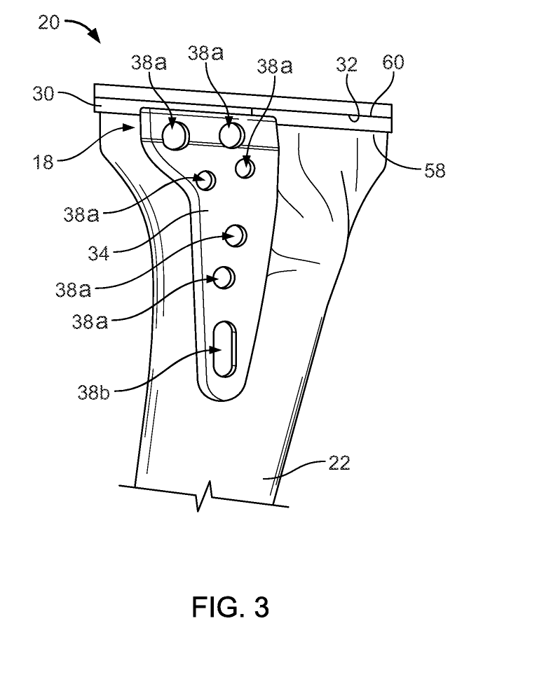

Referring also to FIG. 2, in use, the base 24 of the femoral augment 12 is secured to the femoral component 14, and the fixation portion 28 of the femoral augment 12 is secured to a cortical surface of the femur 16 to aid in firmly fixing the femoral component 14 to the femur 16. Referring also to FIG. 3, in use, the base 30 of the tibial augment 18 is secured to the tibial component 20, and the fixation portion 34 of the tibial augment 18 is secured to a cortical surface of the tibia 22 to aid in firmly fixing the tibial component 20 to the tibia 22. The fixation portion 28 contacts, for example, a medial and/or a lateral side of the femur 16, and the fixation portion 34 contacts, for example, an anterior side of the tibia 22. The fixation portions 28, 34 are, for example, cemented to the cortical surfaces of the femur 16 and the tibia 22, respectively. Alternatively, or additionally, the fixation portions 28, 34 include one or more through holes, such as through holes 36a-c, 38a-b, respectively, for receiving fasteners (not shown) that attach the fixation portions 28, 34 to the femur 16 and the tibia 22, respectively.

The through holes 36a-c, 38a-b can include any type of openings, such as, but not limited to, threaded, non-threaded, partially threaded, polyaxial, tabbed, and finned openings. Inner walls of the through holes 36, 38 can be parallel, converging, diverging, or any combination thereof. Additionally, the through holes 36a-c, 38a-b can be circular holes, slots, or combination locked/non-locked slots, “snowman,” or “light-bulb” shaped. Examples of various applicable through holes are described in Published Patent Applications US2009/0143824, SYSTEMS AND METHODS FOR USING POLYAXIAL PLATES, US2010/0331843, ORTHOPAEDIC IMPLANT AND FASTENER ASSEMBLY, and WO2011/002903, ORTHOPAEDIC IMPLANT AND FASTENER ASSEMBLY, hereby incorporated by reference in their entirety. The fasteners (not shown) can be locking or non-locking, including, but not limited to, cortex, compression, osteopenic, cancellous, lag, lag with cancellous threads, rafter, tapered, and polyaxial screws. Depending on the combination of openings and fasteners used, the through holes 36a-c, 38a-b can provide locking fixation, non-locking fixation, compression, dynamization, or the like to a portion of bone proximate the fixation portions 28, 34.

The fixation portions 28, 34 can be attached to the cortical surface of the bone by inserting one or more fasteners (not shown) through the one or more openings 36a-c, 38a-b into the bone. During surgery, the surgeon creates a skin opening near the knee to insert the knee implant 10. Depending on the size of the skin opening, the size of the fixation portions of the augments, or both, the surgeon can attach the fixation portions of the augments to the bone in a number of ways. For example, a percutaneous approach includes inserting the fastener through a portion of skin covering the fixation portion, through the fixation portion, and into the bone. The location for inserting the fastener through the skin is determined, for example, by palpating the skin by hand and feeling for the openings in the fixation portion. Alternatively, or additionally, a targeting device, such as a jig, a radiolucent drill guide and targeter, or the Smith & Nephew SureShot™ targeting system, can be used to determine the location for inserting the fastener. The surgeon can use a minimally invasive approach by making a stab incision in the skin covering the fixation portion prior to inserting the fastener.

In some cases, the fixation portions 28, 34 of the augments are attached to the cortical surface of the bone through a non-percutaneous approach. For example, the fastener is inserted through the skin opening through which the implant was inserted, and through the one or more openings 36a-c, 38a-b into the bone. In some cases, the skin opening is enlarged, for example, by an incision, to improve accessibility to the openings in the fixation portion.

If the fixation portions 28, 34 were, for example, 10 hole plates that extended to the mid-shaft of the bone, percutaneous and targeted would generally be the preferred approach. If the fixation portions 28, 34 were, for example, shorter 3 hole plates, a targeted approach would generally be used if the holes were locking holes and the screw angle entry were important, for example, if the screws were converging or diverging within the distal end of the femur.

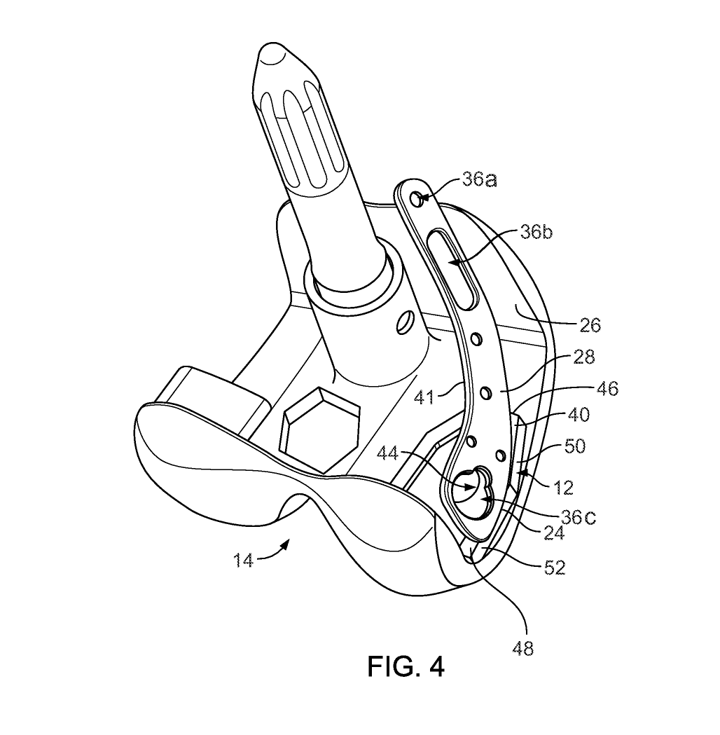

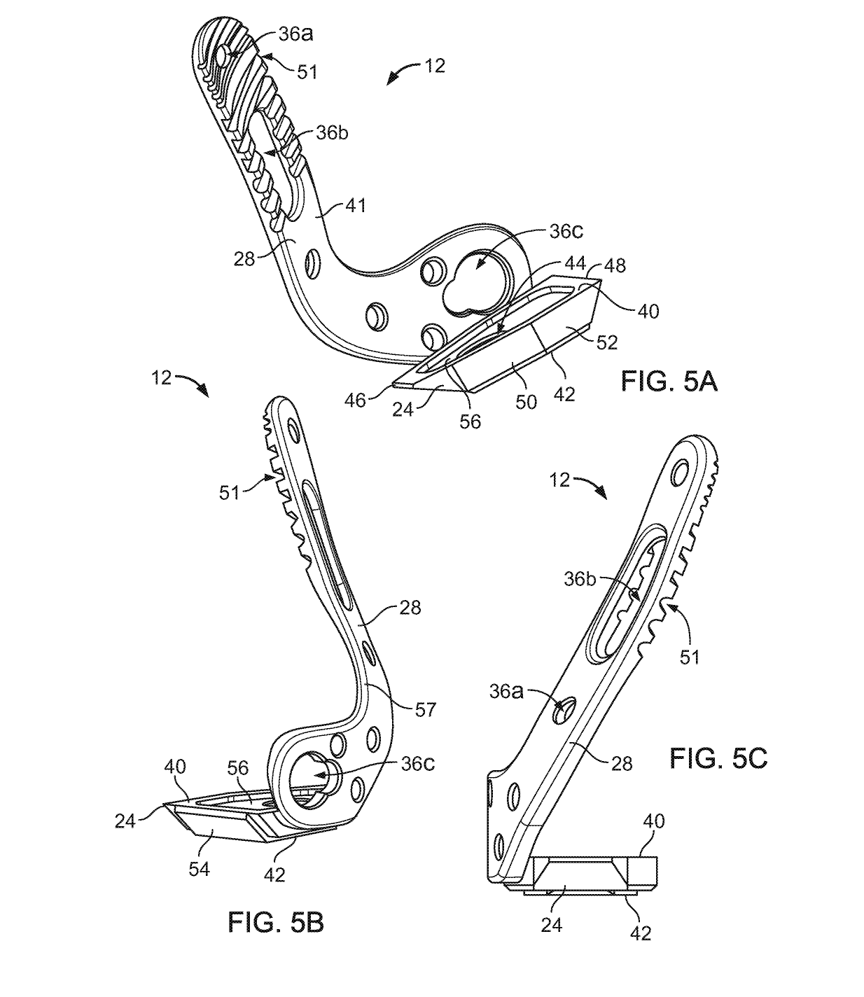

Referring to FIGS. 4 and 5A-5C, the base 24 of the femoral augment 12 includes a bone contacting surface 40 that in use contacts a cut surface of an articular end of the femur 16 (FIG. 2), and an implant contacting surface 42 that in use contacts the inner surface 26 of the femoral component 14. The surgeon selects a femoral augment 12 having a shape and size that compensates for a bone defect, for example, damaged bone the surgeon has removed from the femur. Prior to fixation of the femoral augment 12, the surgeon typically prepares the femur by creating a distal femoral cut that is offset on the affected side of the bone to accept the augment.

The fixation portion 28 includes a bone contacting surface 41 that, in the illustrated implementation, generally conforms to an anatomical shape of the femur 16. In some cases, the fixation portion 28 can be patient matched to the anatomical shape of the patient's femur 16. In a gross anatomic approach of patient matching, the fixation portion 28 is manufactured to correspond to bone shapes of the general population. In a custom anatomic approach of patient matching, the fixation portion 28 is custom made pre-operatively for a particular patient based on, for example, the patient's CT or MRI data, or the fixation portion can be custom made intra-operatively. The femoral augment 12 can be positioned laterally and/or medially on the femoral component 14.

The base 24 of the femoral augment 12 includes one or more through holes 44 for receiving fasteners (not shown) that attach the base 24 to the femoral component 14. The through holes 44 can be locking or non-locking. Additionally, the through holes 44 can receive a taper lock fastener. Alternatively, or additionally, the base 24 of the femoral augment 12 can be cemented to the femoral component 14. The femoral augment 12 and the femoral component 14 can be attached by a snap-in connection. The base 24 defines a relief section 56 that provides clearance for the head of an inserted fastener such that the fastener head lies flush with the bone contacting surface 40 of the base 24 and can additionally contain any overflow of cement that may be present. In use, after securing the base 24 of the femoral augment 12 to the inner surface 26 of the femoral component 14, the surgeon attaches the femoral component 14 to the cut surface of the femur 16 and secures the fixation portion 28 of the femoral augment 12 to the cortical surface of the femur 16, as discussed above, to provide additional stability to the implant 10. The bone contacting surface 40 and the implant contacting surface 42 of the base 24 are generally flat and parallel to each other, as shown in FIGS. 5A-5C.

In the illustrated implementation, the fixation portion 28 includes guide pin holes 36a, a compression slot 36b, and a lag screw opening 36c. The lag screw opening 36c, which can be “snowman” or “light-bulb” shaped, is designed to receive a lag/compression screw combination. While being driven into the bone, the compression screw engages the lag screw in a manner that applies compression to the bone. Additionally, or alternatively, other types of openings, such as those mentioned above for the fixation portion 28, can be used. The fixation portion 28 is generally a thin and elongated strip or plate and can include portions that are flat, straight, curved in-plane, curved out-of-plane, or twisted to generally follow a contour of the shape of the femur 16 at the implant location. For example, the fixation portion 28 is generally L-shaped and bends inwardly at a region 57 to match the curvature of the femur. In some cases, the thickness, width, or both of the fixation portions 28 can vary along a length of the fixation portion 28. The through holes 36a-c can be positioned anywhere along the length of the fixation portion 28 as required.

Bone ingrowth following implantation can be enhanced by modifying the bone contacting surface 41 of the fixation portion 28. For example, scallops 51 can be added to the surface 41 to enhance bone growth. Alternatively, or additionally, the bone contacting surface 41 can be porous, ridged, and/or coated with an ingrowth promoting material such as HA, BMP, or silver.

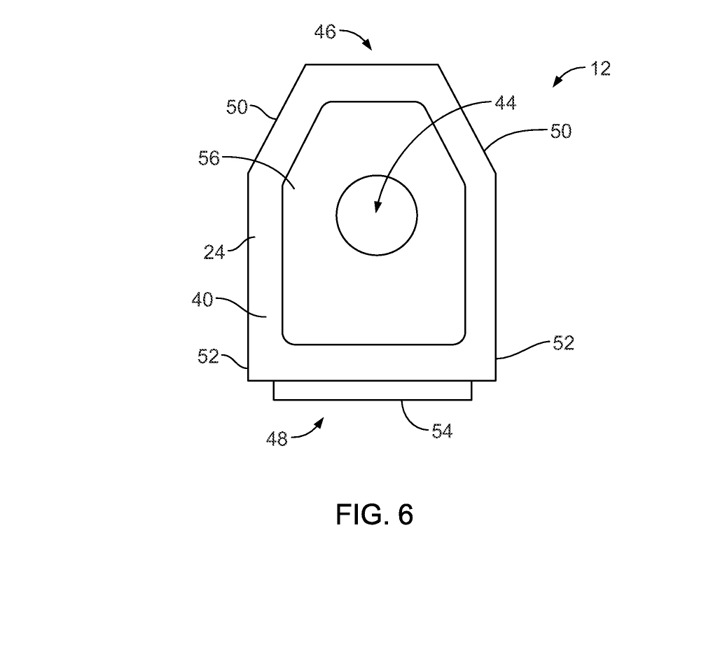

Referring also to FIG. 6, the base 24 is sized to be flush with the inner surface of the femoral component 14. The base 24 has, for example, an anterior portion 46 that is pyramid or wedge shaped formed by two converging side walls 50, and a posterior portion 48 that includes two substantially parallel side walls 52. The posterior portion 48 can include a sloped portion 54 that conforms to the shape of the femoral component 14. The fixation portion 28 (FIGS. 5A-5C) can be attached to one of the side walls 52. Alternatively, the femoral augment 12 can be formed from a single piece of material.

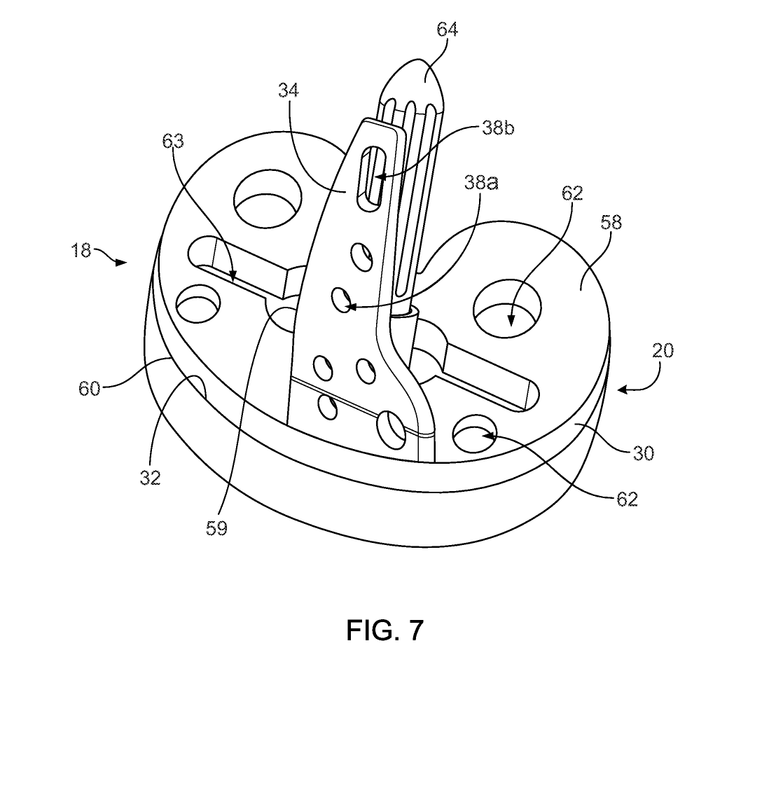

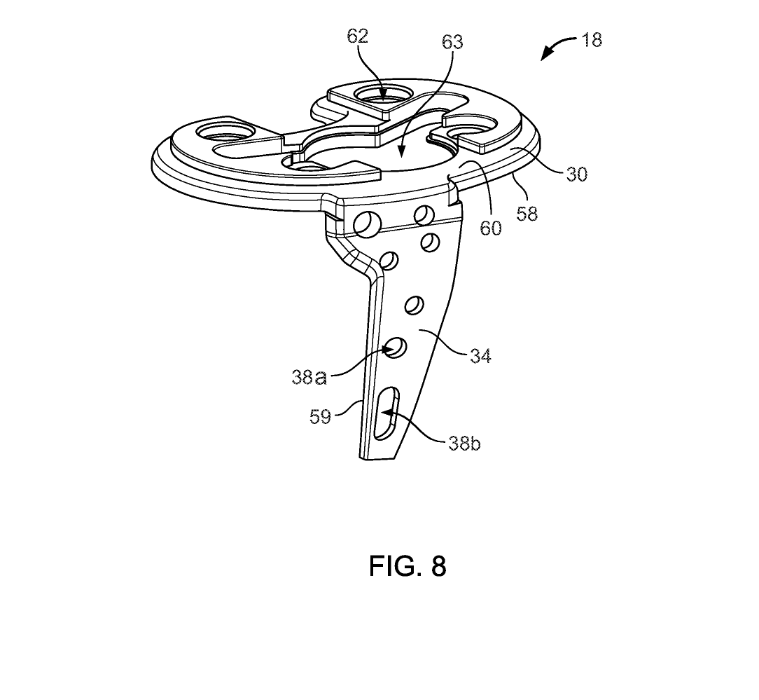

Referring to FIGS. 7 and 8, the base 30 of the tibial augment 18 includes a bone contacting surface 58 that in use contacts the cut surface of an articular end of the tibia 22 (FIG. 3), and an implant contacting surface 60 that in use contacts the face 32 of the tibial component 20. The shape and configuration of the base 30 of the tibial augment 18 generally match the shape and configuration of the face 32 of the tibial component 20. The surgeon selects a tibial augment 18 having a shape and size that compensates for a bone defect, for example, damaged bone the surgeon has removed from the tibia, to maintain the joint line and the position of the collateral ligaments. Prior to fixation of the tibial augment 18, the surgeon typically prepares the tibia by adjusting the initial resection to accept the augment.

The fixation portion 34 includes a bone contacting surface 59 that generally conforms to the anatomical shape of the tibia 22. In some cases, the fixation portion 34 is patient matched to the anatomical shape of the patient's tibia 22. In a gross anatomic approach of patient matching, the fixation portion 34 is manufactured to correspond to bone shapes of the general population. In a custom anatomic approach of patient matching, the fixation portion 34 is custom made for a particular patient based on, for example, the patient's CT or MRI data.

The base 30 of the tibial augment 18 includes one or more through holes 62 for receiving fasteners (not shown) that attach the base 30 to the tibial component 20. The through holes 62 can be locking or non-locking. Alternatively, the base 30 of the tibial augment 18 can be cemented to the tibial component 20. The base also includes an opening 63 through which a stem 64 of the tibial component can pass.

The fixation portion 34 of the tibial augment 18 includes circular openings 38a and a slot 38b. The circular openings 38a can be locking holes that receive a threaded fastener for locking fixation. The slot 38b can receive a partially threaded fastener for sliding fixation. Additionally, or alternatively, other types of openings, such as those mentioned above, can be used. The fixation portion 34 is generally a thin and elongated plate that in use extends distally from a proximal region of the tibial component 20 on the anterior side of the tibia 22. The fixation portion 34 can include portions that are flat, straight, curved in-plane, curved out-of-plane, or twisted and is generally shaped to follow a contour of the tibia 22 at the implant location. In some cases, a thickness, width, or both of the fixation portions 34 can vary along a length of the fixation portion 34. For example, a proximal region of the fixation portion 28 can be wider than a distal region. The through holes 38a-b can be positioned anywhere along the length of the fixation portion 34 as required.

In use, after securing the base 30 of the tibial augment 18 to the face 32 of the tibial component 20, the surgeon attaches the tibial component 20 to the cut surface of the tibia 22 and secures the fixation portion 34 of the tibial augment 20 to the cortical surface of the tibia 22 to provide additional stability to the tibial component 18.

The bone contacting surface 58 and the implant contacting surface 60 of the base 30 are generally flat and parallel to each other. Alternatively, the bone contacting surface 58 and the implant contacting surface 60 can converge towards each other to create a base 30 with, for example, a wedge-like side profile. The tibial augment 18 is formed from a single piece of material, or the base 30 and the fixation portion 34 can be formed separately and attached to each other.

The femoral augment 12 and the tibial augment 18 can be formed from any biocompatible material or a combination of biocompatible materials, such as certain metal alloys and polymers. Additionally, the femoral augment 12 and the tibial augment 18 can be formed using any manufacturing techniques, such as selective laser sintering (SLS).

Other implementations are within the scope of the following claims.

For example, the size and shape of the femoral augment 12 and the tibial augment 18 can be varied to provide the desired patient fit.

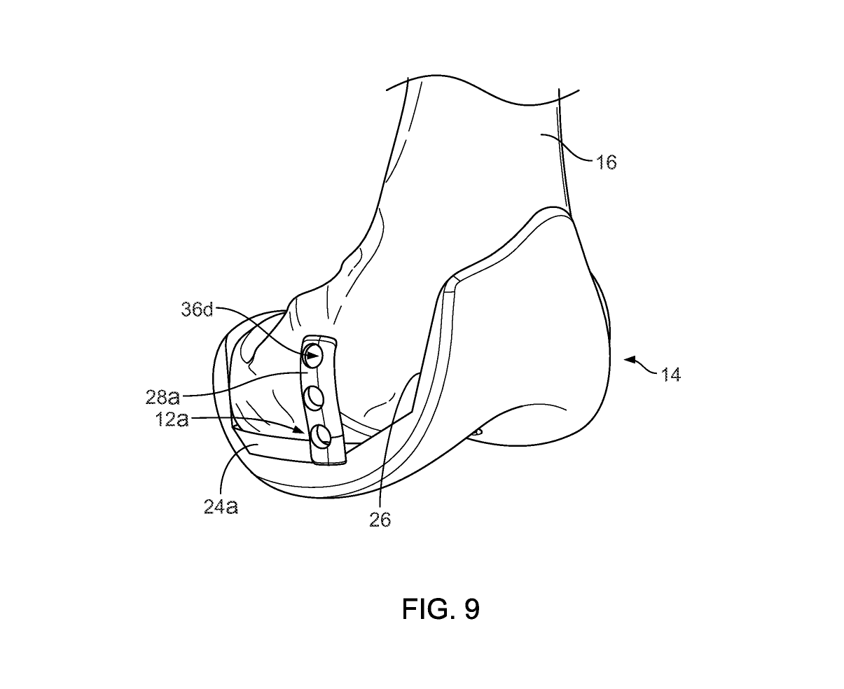

Referring to FIG. 9, in an alternative implementation, a femoral augment 12a includes a base 24a and a fixation portion 28a. The fixation portion 28a is generally rectangular and extends proximally from a side portion of the base 24a. The fixation portion 28a includes portions that are curved to match a local curvature of the bone. One or more, for example, three, through holes 36d are positioned along a length of the fixation portion 28a. In this implementation, the fixation portion 28a is generally shorter and straighter than that of the fixation portion 28 described above.

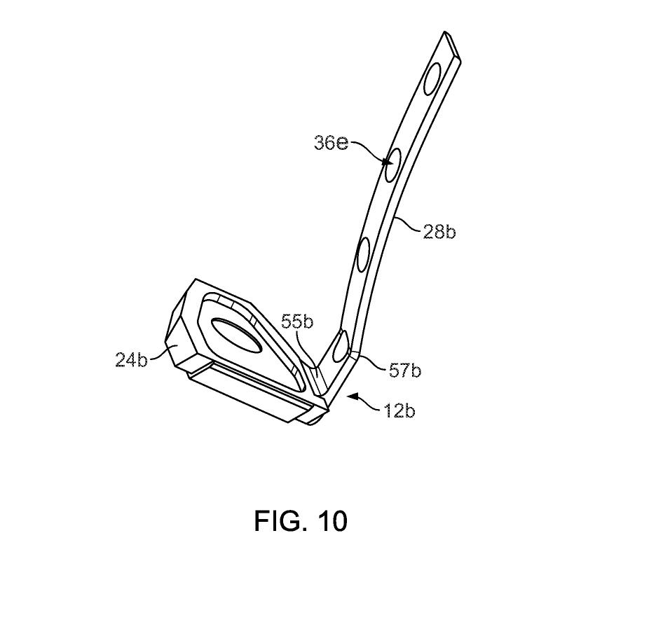

Referring to FIG. 10, in another implementation, a femoral augment 12b includes a base 24b, a fixation portion 28b, and an extension portion 55b connecting the femoral augment 12b to the fixation portion 28b. One or more, for example, four, through holes 36e are positioned along a length of the fixation portion 28b. Additionally, the fixation portion 28b includes a bend 57b that conforms to the surface of the bone.

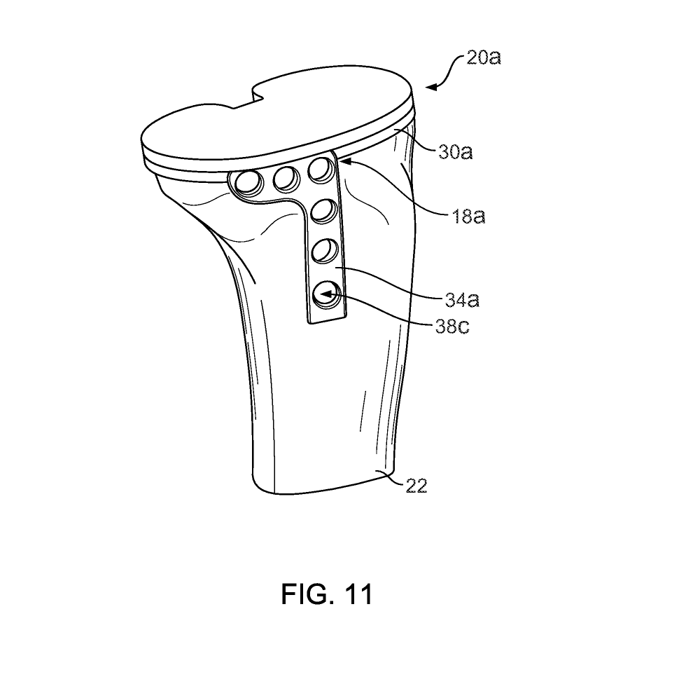

Referring to FIG. 11, an implementation of a tibial augment 18a includes a base 30a and a fixation portion 34a. The fixation portion 34a is L-shaped, having a horizontal portion that extends mediolaterally and a vertical portion that extends distally. One or more, for example seven, through holes 38c are positioned along the horizontal and vertical portions of the L-shaped fixation portion 34a.

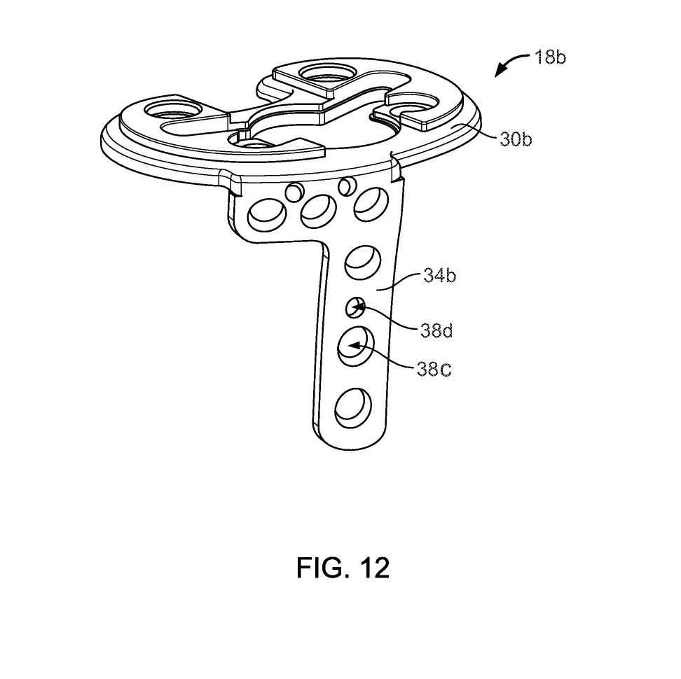

As shown in FIG. 12, a tibial augment 18b includes a base 30b and a fixation portion 34b, and the fixation portion 34b includes one or more, for example, six, through holes 38c and one or more, for example, three, pilot holes 38d.

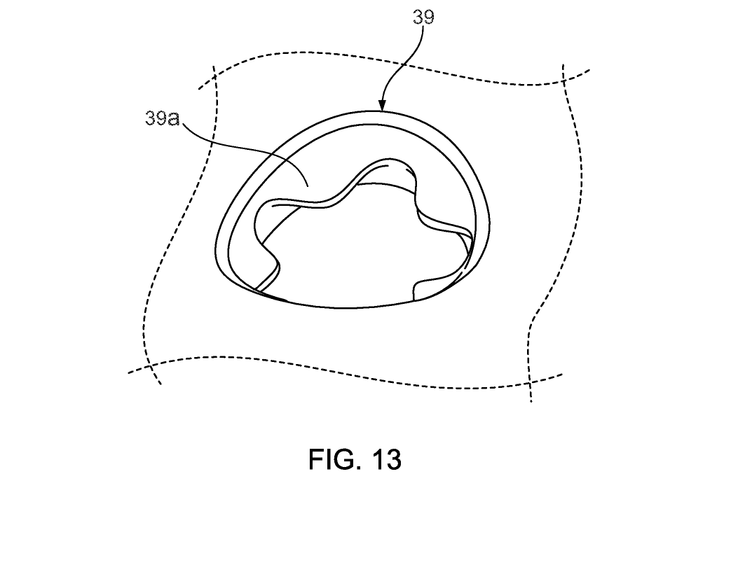

Referring to FIG. 13, through holes 36a-c, 38a-b can include a polyaxial opening, such as a finned opening 39. The finned opening 39 includes a series of concavely indented, inwardly protruding fins 39a. The fins 39a allow a fastener (not shown) to be inserted at any angle relative to a central axis defined by the opening 39.

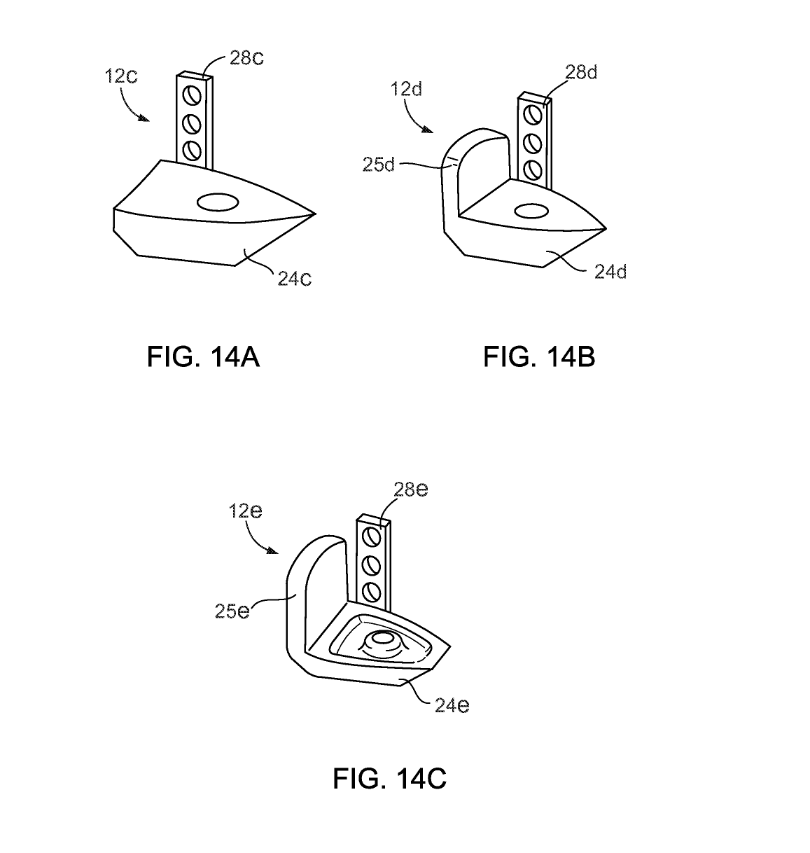

Referring to FIGS. 14A-14C, the base of the femoral augment can vary in shape depending on the femoral implant the base is being used with and the condition of the articular surface of the femur. As shown in FIG. 14A, a base 24c of a femoral augment 12c has a pointed tip in contrast to a relatively flat tip of the base 24 (FIG. 6). Additionally, the base 24c is thicker than the base 24 of the femoral augment 12 to provide, for example, a desired proximodistal positioning of the femoral implant with respect to the articular surface of the femur. As shown in FIG. 14B, a base 24d of a femoral augment 12d includes a posterior portion 25d that extends proximally from the base 24d. The posterior portion 25d can be used, for example, to provide a desired anteroposterior positioning of the femoral implant with respect to the articular surface of the femur. As shown in FIG. 14C, a femoral augment 12e has a base 24e and a posterior portion 25e. Similar to the base 24 of the femoral augment 12 (FIG. 5A), the base 24e has a flat tip and is thinner than the bases 24c, 24d.

Rather than covering the entire face 32 of the tibial component 20, the base 30 can be smaller than the tibial component such that the base 30 covers a smaller portion of the face 32 of the tibial component 20, such as a lateral portion of the face 32. In some cases, more than one tibial augment 18 can be used on a single tibial component 20.

The femoral augment 12 and the tibial augment 18 can be secured to the femoral component 14 and the tibial component 20, respectively, and/or to the cortical surface of the femur 16 and the tibia 22, respectively, by using various types of fasteners including, but not limited to, screws, bolts, pins, and rivets. Alternatively, or additionally, the femoral augment 12 and the tibial augment 18 can be held in place by friction or other mechanical means. For example, the femoral augment 12 and the tibial augment 18 can be cemented to the femoral component 14 and the tibial component 20, respectively, or to the cortical surfaces of the femur 16 and the tibia 22, respectively. In some cases, the femoral augment 12 and the tibial augment 18, respectively, can be integrated with the femoral component 14 and the tibial component 20. In some cases, the fixation portions 28, 34 can be attached directly to the femoral component 14 and the tibial component 20, respectively.

The fixation portions 28, 34 can be attached to and/or detached from the base 24, 30, respectively, following installation of the implant 10. In some cases, the fixation portions 28, 34 can be bent after installation of the implant 10 to more securely contact the cortical surface of bone. The base 24, 30, respectively, of the femoral augment 12 and the tibial augment 18 can include more than one fixation portions 28, 34, respectively.

The surgeon can first attach the femoral augment 12 and the tibial augment 18 to the femur 16 and the tibia 22, respectively, before attaching the femoral augment 12 and tibial augment 18 to the femoral component 14 and the tibial component 18, respectively.

All bone contacting surfaces can include features such as described above to enhance bone ingrowth.

The assembly of the augments with the femoral and tibial components can occur as a back table assembly.

What is claimed is:

1. An implant augment, comprising:
an augment portion having a first surface for contacting a cut surface of an articular end of a bone and a second surface for contacting an implant; and
a fixation portion attached to the augment portion and extending from the augment portion to contact a cortical surface of a shaft of the bone.

2. The implant augment of claim 1, wherein the fixation portion defines one or more through holes for receiving fasteners.

3. The implant augment of claim 2, wherein the holes comprise locking holes.

4. The implant augment of claim 2, wherein the holes comprise non-locking holes.

5. The implant augment of claim 1, wherein the fixation portion is patient matched to an anatomical shape of a patient's bone.

6. The implant augment of claim 1, wherein the fixation portion includes a porous in-growth surface.

7. The implant augment of claim 1 comprising a femoral augment.

8. The implant augment of claim 1 comprising a tibial augment.

9. The implant augment of claim 1, wherein the augment portion defines a through hole.

10. The implant augment of claim 1, wherein the augment portion is formed from a biocompatible material.

11. The implant augment of claim 1, wherein the fixation portion includes a surface that enables cement adherence between the fixation portion and the cortical surface of the bone.

12. A surgical method, comprising:
positioning an augment portion of an implant augment on a cut surface of an articular end of a bone; and
fixing a fixation portion of the implant augment to a cortical surface of a shaft of the bone.

13. The surgical method of claim 12, wherein fixing the fixation portion of the implant augment includes inserting one or more fasteners through one or more openings defined in the fixation portion into the bone.

14. The surgical method of claim 12 comprising detaching the fixation portion from the augment portion following installation of the implant augment.

15. The surgical method of claim 12, wherein the implant augment comprises a femoral augment or a tibial augment.
